(19) United States
(12) Patent Application Publication
(10) Pub. No.: US 2024/0180726 A1
(43) Pub. Date: Jun. 6, 2024

(54) ADJUSTABLE UPPER LIMB PROSTHETIC GRIPPING DEVICE

(71) Applicant: Form5 Prosthetics Inc., New Albany, OH (US)

(72) Inventors: Aaron Westbrook, New Albany, OH (US); Lindsey Austin, Lewis Center, OH (US); Joshua T. Johnson, Waukesha, WI (US); Alanna Ho, Windsor Mill, MD (US)

(21) Appl. No.: 18/530,781

(22) Filed: Dec. 6, 2023

Related U.S. Application Data
(60) Provisional application No. 63/430,523, filed on Dec. 6, 2022.

(51) Int. Cl. A61F 2/80; A61F 2/50; A61F 2/58

(52) U.S. Cl. CPC A61F 2/80; A61F 2/5044; A61F 2/583; A61F 2002/5083; A61F 2002/7862

(57) ABSTRACT

A prosthetic device is described for assisting amputees in touching, gripping, and repositioning various objects. In one example of the device of the present invention, the device has a first socket for receiving a portion of a residual arm below an elbow, and a second socket attached to and/or rotatable with respect to the first socket. The second socket may receive a portion of the residual arm above the elbow. Movement of the residual arm within the device may define movement of the device, such as, for example, gripping movement of a prosthetic hand of the device.

### CROSS-REFERENCE TO RELATED APPLICATIONS

This non-provisional patent application claims priority to U.S. Provisional Application Ser. No. 63/430,523, filed on Dec. 6, 2022, the disclosure of which is incorporated by reference as if fully recited herein.

### TECHNICAL FIELD

The present invention relates to a prosthetic device that permits an individual missing a portion of at least one arm to touch, grip, reposition, some combination thereof, or the like any number of different objects by engaging the prosthetic device with a residual arm. An example embodiment includes a first socket adapted to receive a portion of a residual arm below an elbow, and a second socket rotatably attached to the first socket, the second socket adapted to receive a portion of the residual arm above the elbow, and permit movement of the residual arm to define movement of the device, including, for example, gripping of a prosthetic hand of the device.

### BACKGROUND AND SUMMARY OF THE INVENTION

Many humans often utilize one or both hands and/or lower arm(s) for various actions involving touching, gripping, object repositioning, and the like. For example, many humans often use one or both hands to grasp and reposition objects. Traditionally, engagement in these actions has often been difficult for arm amputees, individuals having congenital arm anomalies, and the like. Although certain known prosthetic devices may enable arm amputees, individuals having congenital arm anomalies, and the like to perform some of these actions, known prosthetic devices are often expensive, uncomfortable, lack adjustable gripping magnitude (e.g., higher gripping magnitude indicates more significant gripping force applied to an object), lack provided user control of device rotation, and lack adaptability for any number of different gripping attachments. Thus, there is an unmet need in the prior art to provide a comfortable, cost-effective prosthetic device that permits an individual missing a portion of at least one arm to adjust device gripping magnitude, control rotation of the device, and attach any number of different gripping attachments to the device (e.g., different prosthetic hands, grasping devices, and the like).

This and other unmet needs of the prior art are addressed by an adjustable prosthetic gripping device as described in more detail below, as well as by a method of assembling said adjustable prosthetic gripping device.

In one exemplary embodiment, an adjustable prosthetic gripping device includes an upper and lower socket, each rotatable with respect to the other, and a socket connector of the lower socket for receiving any number of different attachments. A string may establish mechanical communication between the lower socket and an exemplary attachment. Tension of the string may be adjusted to regulate gripping magnitude of the attachment.

In another exemplary embodiment, a method for assembling an adjustable prosthetic gripping device involves providing an upper and lower socket rotatable with respect to one another, and configuring the lower socket to receive any number of different attachments. A string may be provided to establish mechanical communication between the lower socket and an exemplary attachment. The device may be configured to permit user adjustment of string tension for regulating gripping magnitude of the attachment.

Exemplary embodiments may be useful for permitting an individual missing a portion of at least one arm to engage in any number of different actions involving touching, gripping, object repositioning, some combination thereof, or the like. A single exemplary device may be used, but in certain cases, more than one exemplary device may be used, such as when, e.g., an individual is missing portions of both arms.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 is a perspective view of an exemplary adjustable prosthetic gripping device in an extended position, according to an exemplary embodiment of the present invention;

FIG. 2 is a side perspective view of the exemplary device of FIG. 1;

FIG. 3 is a perspective view of an exemplary gripping hand rotatably attached to an exemplary lower socket of the device of FIG. 1;

FIG. 4 is another perspective view of the exemplary device of FIG. 1;

FIG. 5 is yet another perspective view of the exemplary device of FIG. 1;

FIG. 6 is a perspective view of an alternative exemplary adjustable prosthetic gripping device in an extended position;

FIG. 7 is a perspective view of the exemplary device of FIG. 6 in a bent position;

FIG. 8 is a perspective view of an exemplary gripping hand being rotated with respect to an exemplary lower socket of the device of FIG. 6; and

FIG. 9 is a perspective view of an exemplary grip string tension adjustment knob in accordance with an exemplary embodiment of the present invention.

### DETAILED DESCRIPTION OF EXEMPLARY EMBODIMENTS

Various embodiments of the present invention will now be described in detail with reference to the accompanying drawings. In the following description, specific details such as detailed configurations and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

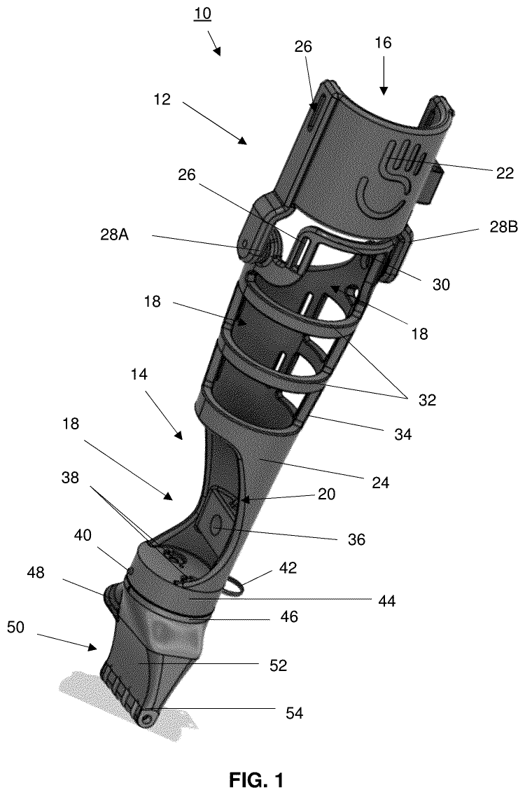

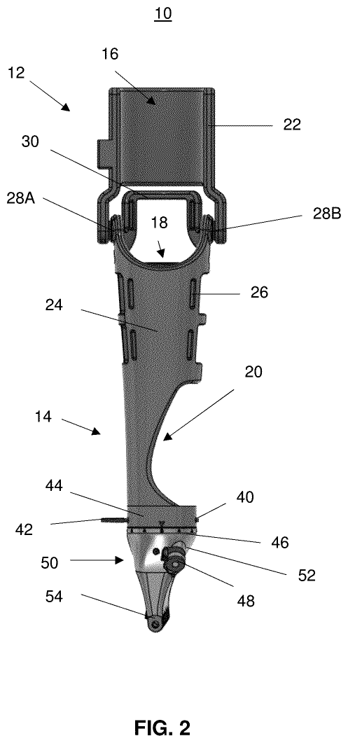

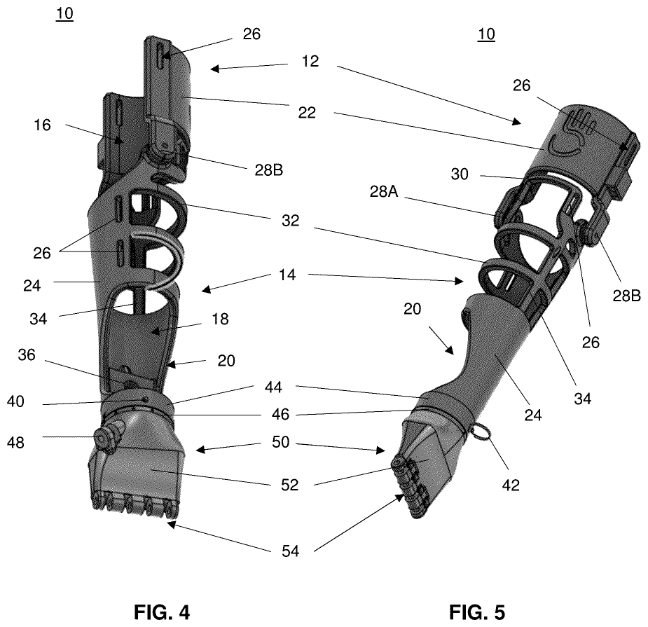

Referring initially to FIGS. 1-5, the device 10 may comprise a lower socket 14 adapted to receive and secure a first portion of a residual arm in at least part of a receptacle 18 of the lower socket 14. The first portion may be located substantially below an elbow, such that the lower socket 14 may at least partially surround and secure the residual arm proximate to at least a portion of the radius and/or ulna thereof. A residual arm is defined herein as a human arm lacking at least a portion of at least one typical arm bone. A residual arm may include, for example, an arm that has had a portion thereof amputated beyond the elbow, such that a user still has the elbow of the arm, but not the hand. As another non-limiting example, a residual arm may include an arm that has been amputated above the elbow, such that the user still has at least a portion of the humerus of the arm, but not the elbow thereof. An exemplary device body may be assembled in any number of different shapes and/or sizes to accommodate any number of different arm amputees or individuals having congenital arm anomalies, regardless of residual arm shape.

The device 10 may further comprise an upper socket 12 adapted to receive and secure a second portion of a residual arm in at least part of a receptacle 16 of the upper socket 12. The second portion may be located substantially above the elbow, such that the upper socket 12 may at least partially surround and secure the residual arm proximate to at least a portion of the humerus thereof. The upper socket 12 may be attached to and rotatable with respect to the lower socket 14, and the lower socket 14 may be attached to and rotatable with respect to the upper socket 12. The lower socket 14 may include a top edge 30. The top edge 30 may be configured to surround an entry and exit region for positioning the first portion of the residual arm in the lower socket 14, and for removing the first portion of the residual arm from the lower socket 14.

In this particular embodiment, a portion of the top edge 30 proximate to top edge links 28A-B is curved below the remainder of the top edge. The curved shape of the top edge 30 may provide for ease of positioning the residual arm in the device 10 and for ease of removing the residual arm from the device 10 without compromising securement of the lower socket 14 to the first portion of the residual arm while the device 10 is in use. A strap (not shown) attached to the device 10 by way of apertures (e.g., 26 in FIG. 1) in the top edge 30 may close around the residual arm to promote securement of the lower socket 14 to the first portion of the residual arm. The curved shape of the top edge 30 in FIGS. 1-5 is merely illustrative. Any number of top edge configurations, including, e.g., straight slope top edge configurations, may be employed.

In this particular embodiment, the top edge links 28A-B are included to permit rotation of the upper socket 12 and the lower socket 14 with respect to one another. The top edge links 28A-B may define forearm links configured to establish connection between the lower socket 14 and the upper socket 12, and each the lower socket 14 and the upper socket 12 may be rotated about said forearm links. Any number of different fasteners, clips, hinges, other mechanical joints, some combination thereof, or the like may be employed to establish rotatable connection between an exemplary lower socket (e.g., 14) and an exemplary upper socket (e.g., 12). Movement of the residual arm (e.g., bending movement) may define rotation of the upper socket 12 and the lower socket 14 with respect to one another. The permitted degrees of freedom of said rotation may be varied without departing from the scope of the present invention. Side edge regions of a body 22 of the upper socket 12 may be slightly elevated beyond the remainder of the body 22 to provide for ease of connecting the upper socket 12 to the lower socket 14, although such is not required.

The body 22 of the upper socket 12 and body 24 of the lower socket 14 may each comprise 3D-printed, substantially plastic material. Exemplary 3D printing may involve resins, laser sintering, some combination thereof, or the like. Any number of different materials and/or methods may be employed for assembling the features described herein. A portion of the lower socket 14 proximate to an end-region 44 (also referred to herein as “socket connector”) thereof may comprise substantially curved edges sloping downward into the body 24 of the lower socket 14 to define an open area 20 of the lower socket 14. The open area 20 may promote airflow at and heat escape from the receptacle 18 of the lower socket 14 to, e.g., promote user comfort. If airflow at and heat escape from the receptacle 18 were substantially restricted, a user might experience discomfort related to overheating of the residual arm caused by heat being trapped within the body 24 of the lower socket 14. Said overheating might result in sweating at skin surfaces of the residual arm, which might in turn result in chafing. Airflow at and heat escape from the receptacle 16 of the upper socket 12 may also be promoted by maintaining substantially open space opposite of the curved body 22 of the upper socket 12. Said substantially open space opposite of the curved body 22 of the upper socket 12 may further promote ease of positioning the second portion of the residual arm in the device 10. The aforementioned substantially open space and the open area 20 may be varied in shape, size and configuration.

The open area 20 of the lower socket 14 may also permit user access to various interior components of the lower socket 14. Interior components may include by way of example and not limitation, fasteners (e.g., 38) adapted to affix various components of the device 10 to one another, apertures (e.g., 36) adapted to permit access to a tension string (not shown) of the device 10, and the like. Although fasteners (e.g., 36) are illustrated herein, any number of different connection mechanisms and/or materials (e.g., clips, fasteners, joints, bolts, some combination thereof, or the like) may be substituted for one another. The open area 20 of the lower socket 14 may further promote consistent air pressure throughout the device. Positioned between the open area 20 of the lower socket 14 and the upper socket 12 may be a number of curved beams 32 and columns 34 of the lower socket 14. The curved beams 32 and columns 34 may provide structural security of the lower socket while also permitting airflow at and heat escape from the region between the open area 20 of the lower socket 14 and the upper socket 12. Said permitted airflow and heat escape may promote user comfort and consistent air pressure throughout the device. The numbers, shapes, configurations, and the like of the various beams and columns may be varied.

A number of apertures 26 may be included at various locations across the upper socket 12 and lower socket 14 to permit straps (not shown) to be linked thereto. Said straps may promote securement of the device 10 to a residual arm. The number, shape and size of apertures (e.g., 26) may be varied. Still referring to FIGS. 1-5, the socket connector 44 may be adapted to receive an attachment (e.g., a prosthetic hand 50). In this particular embodiment, the prosthetic hand 50 comprises a palm region 52, thumb joint 48, and finger joints 54. It will be apparent to one of ordinary skill in the art, however, that any number of different attachments (e.g., grasping, clamping, gripping, some combination thereof, or the like components) may be connected to an exemplary socket connector (e.g., 44). As a specific, non-limiting example, a climbing hook may be attached to an exemplary socket connector. Additionally, any number of different prosthetic hands may be connected to an exemplary socket connector.

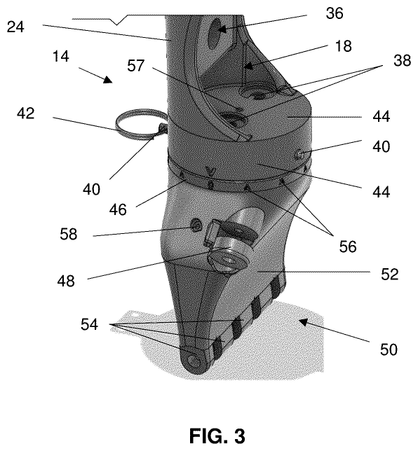

The attachment (e.g., prosthetic hand 50) may be attached to the socket connector 44 by aligning apertures (not shown) of a connection portion 46 of the attachment (e.g., prosthetic hand 50) with apertures (not shown) of the socket connector 44, and positioning a pin 40 through certain apertures of each the socket connector 44 and the connection portion 46 of the attachment (e.g., prosthetic hand 50). The pin 40 may be configured to affix the attachment (e.g., prosthetic hand 50) to the lower socket 14. Specifically, in this particular embodiment, the pin 40 may be inserted into each of a first aperture of the socket connector 44 and a first aperture of the connection portion 46, and then each of a second aperture of the connection portion 46 and a second aperture of the socket connector 44 both opposite of the first apertures, to secure the attachment. The pin 40 may be removed to permit the attachment (e.g., prosthetic hand 50) to be removed from and/or rotated with respect to the lower socket 14. A ring 42 may be linked to the pin 40 to permit a user to insert and/or remove the pin 40 with respect to said certain apertures. Referring specifically to FIG. 3, the apertures of the connection portion 46 of the attachment (e.g., prosthetic hand 50) may be spaced apart in 30-degree increments, such that the attachment (e.g., prosthetic hand 50) may be rotated and secured in 30-degree increments across a 360-degree field of rotation. The increments may be indicated to a user for ease of alignment by each of a plurality of indicators 56. The 30-degree increments illustrated are in no way intended to be exhaustive of the scope of the present invention. Any number of different rotation intervals may be provided.

Various features of the attachment (e.g., prosthetic hand 50) may be affixed to one another by fasteners (e.g., 58) and/or any number of other connection mechanisms/materials (e.g., clips, joints, bolts, some combination thereof, or the like). Connection mechanisms/materials may be substituted for one another without departing from the scope of the present invention. A string (not shown) for regulating gripping magnitude of a gripping mechanism of the attachment may be positioned through a string aperture 57 to establish mechanical communication between the string and the attachment (e.g., prosthetic hand 50). A user may move the residual arm to generate tension in the string, wherein tensile force applied from the string on the attachment (e.g., prosthetic hand 50) may cause the attachment to apply gripping force. For example, a user may bend the residual arm to generate tension in the string, wherein tensile force applied from the string on a prosthetic hand 50 causes fingers (not shown) of the prosthetic hand 50 to close, causing the prosthetic hand 50 to grip an object.

Although example embodiments illustrated and described herein involve an upper socket (e.g., 12) and top edge links (e.g., 28A-B) positioned proximate to an elbow, top edge links (e.g., 28A-B) and an upper socket (e.g., 12) are not necessarily required in other embodiments. It is also not necessarily required that a residual arm include an elbow. As a non-limiting example, a user may place a residual arm comprising a portion of a humerus in a single socket having an exemplary socket connector. Furthermore, although example embodiments illustrated and described herein involve a string for regulating attachment gripping, said string is not necessarily required in other embodiments, and may be substituted with other gripping mechanisms and/or components. Also, multiple strings may be employed in other embodiments. Additionally, while certain embodiments described herein involve positioning a pin within apertures to secure an attachment to a socket connector (e.g., 44), a pin is not necessarily required. Any number of different attachment devices, methods, and/or materials may replace and/or supplement example attachment devices, methods, and/or materials illustrated and described herein without necessarily departing from the scope of the present invention.

An exemplary device may be adjusted to receive and secure any number of different sized residual arms. For example, straps may be adapted to secure an exemplary device to any number of different residual arms. In certain exemplary embodiments, a length adjustment mechanism may be provided to permit adjustment of the length of the device (e.g., based on the height of the user, the length and size of the residual arm, some combination thereof, or the like). It will be apparent to one of ordinary skill in the art that there may be any number of different materials and/or methods available for providing length adjustment of an exemplary device.

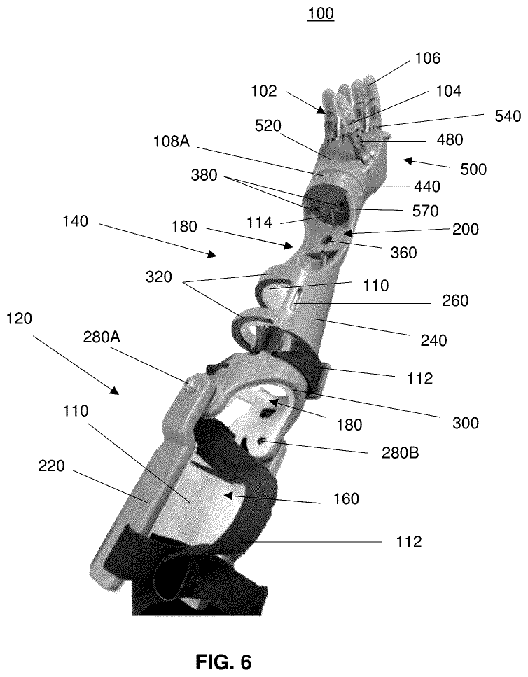

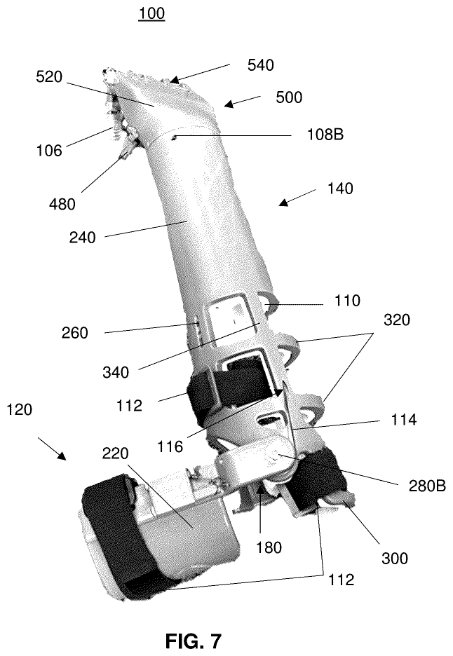

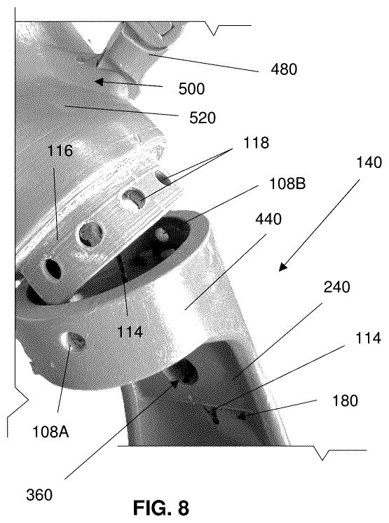

Now referring to FIGS. 6-8, an alternative device 100 may comprise an upper socket 120 having a substantially 3D-printed plastic body 220 and foam 110 interior surrounding a portion of a receptacle 160, and a lower socket 140 having a substantially 3D-printed plastic body 240 and foam 110 interior surrounding a portion of a receptacle 180. Although advantageous, 3D-printed plastic is not necessarily required. In this particular embodiment, straps 112 are configured to secure the exemplary device 100 to any number of different residual arms. The straps 112 may close around a residual arm by way of hook and loop fastening, and/or any number of other strap adjustment techniques for accommodating variations between arms. The straps 112 may be linked to the device 100 by way of positioning a portion of each strap 112 in strap apertures 260 of the device 100. The foam 110 may promote user comfort by cushioning the residual arm with respect to the socket bodies 220, 240 and preventing chafing. Any number of different materials may be employed to define exemplary straps, exemplary foam, and the like. Furthermore, although advantageous, foam and straps are not necessarily required in other embodiments.

The lower socket 140 may include a top edge 300 and top edge links 280A-B. Positioned between the top edge 300 and an open area 200 may be a number of curved beams 320 and columns 340 configured with foam 110 interiors. Interior components including by way of example and not limitation, fasteners (e.g., 380) adapted to affix various components of the device 100 to one another, an aperture (e.g., 360) adapted to permit access to a tension string 114 of the device 100, and the like may be accessed by a user through the open area 200. The string 114 may extend through an aperture 570 of a socket connector 440 to establish mechanical communication between the string 114 and finger 540 and thumb 480 joints proximate to a palm region 520 of a prosthetic hand 500. Tensile force from the string 114 may cause contraction of a thumb 104 and fingers 102 of the prosthetic hand 500. Contraction of a residual arm may increase said tensile force by causing retraction of the string 114. Said contraction may provide gripping of an object. Each of the thumb 104 and fingers 102 may comprise a gripping surface 106 preferably defined by material configured to provide a high friction coefficient. Said gripping surfaces 106 may provide for improved gripping of an object. Any number of different suitable materials for providing a high friction coefficient may be employed. Although advantageous, gripping surfaces (e.g., 106) are not necessarily required in other embodiments.

The string 114 may extend from the aperture 570 to a string adjustment mechanism at or near a top edge link (e.g., 280B), although such is not required. A channel 116 may secure the string 114 along a length of the device 100. The string 114 may extend from the channel 116 past the aperture 360 and through the socket connector 440 to establish mechanical communication between the lower socket 140 and a gripping mechanism (e.g., fingers 102) of an attachment (e.g., prosthetic hand 500). The prosthetic hand 500 may be attached to the socket connector 440 by aligning apertures 118 of a connection portion 116 of the prosthetic hand 500 with apertures 108A-B of the socket connector 440, and then positioning a pin and/or similar locking object (not shown) through each of a first aperture 108A of the socket connector 440 and a first aperture 118 of the connection portion 116, and then each of a second aperture 118 of the connection portion 116 and a second aperture 108B of the socket connector 440, both opposite of the first apertures. The apertures of the connection portion 116 may be spaced apart in 30-degree increments, such that the prosthetic hand 500 may be rotated and secured in 30-degree increments across a 360-degree field of rotation, although such is not required.

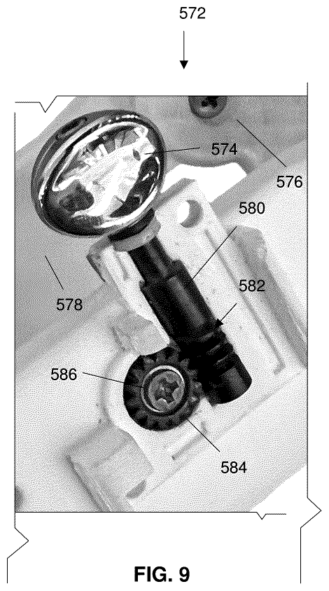

Referring now to FIG. 9, an exemplary device 572 may be configured with a string tension adjustment knob 574 (also referred to herein as “tensioning pin”) positioned on a device body 578, and adapted to regulate tension of an exemplary string in mechanical communication with a gripping attachment. The tensioning pin 574 may be positioned proximate to a forearm link (e.g., 576), although such is not required. The tensioning pin 574 may be configured to regulate motion of a worm gear 580 adjacent thereto and in mechanical communication therewith. Ridges 582 of the worm gear 580 may be configured to engage a receiving gear 584 (e.g., connected to the device body 578 by a fastener 586) to modify tension of the string, wherein the string may be in direct mechanical communication with the receiving gear 584. The tensioning pin 574 may be rotated in a first direction to increase tension in the string, and thus increase gripping magnitude of an attachment of the device 100, and may be rotated in a second direction to decrease tension in the string, and thus decrease gripping magnitude of the attachment. The aforementioned string tension adjustment mechanism is not necessarily required in other embodiments. Any number of different string tension adjustment mechanisms may be employed.

Any embodiment of the present invention may include any of the features of the other embodiments of the present invention. The exemplary embodiments herein disclosed are not intended to be exhaustive or to unnecessarily limit the scope of the invention. Exemplary embodiments were chosen and described in order to explain the principles of the present invention so that others skilled in the art may practice the invention. Exemplary embodiments with respect to residual arms were shown and described herein for illustrative purposes, but it will be apparent to one of ordinary skill in the art that other embodiments may be adapted for use with other body parts without necessarily departing from the scope of the present invention. Having shown and described exemplary embodiments of the present invention, those skilled in the art will realize that many variations and modifications may be made to the described invention. Many variations and modifications will provide the same result and fall within the spirit of the claimed invention. It is the intention, therefore, to limit the invention only as indicated by the scope of the claims.

What is claimed is:

1. An adjustable prosthetic gripping device comprising:
a first socket, adapted to receive and secure a portion of a limb in a receptacle of said first socket;
an attachment;
a socket connector, positioned at a distal end of said first socket;
wherein said attachment is configured to be removably secured to said socket connector at a number of different angles; and
wherein said attachment is configured to receive a tensile force caused by movement of said limb to cause said attachment to apply a gripping force.

2. The device of claim 1, wherein said attachment is a prosthetic hand.

3. The device of claim 1, further comprising a second socket, adapted to receive and secure another portion of said limb in a receptacle of said second socket, wherein said first socket and said second socket are attached to one another and are rotatable with respect to one another, and wherein open space is present opposite of said second socket.

4. The device of claim 1, further comprising a string configured to apply said tensile force.

5. The device of claim 1, wherein a portion of said first socket proximate to said socket connector comprises edges defining an open area of said first socket.

6. The device of claim 5, wherein said edges of said portion of said first socket proximate to said socket connector are substantially curved.

7. The device of claim 1, wherein said attachment comprises a plurality of apertures each configured to be aligned with an aperture of said socket connector to permit a locking object to be positioned through aligned apertures to secure said attachment to said socket connector.

8. The device of claim 1, wherein said first socket comprises a foam interior.

9. An adjustable prosthetic gripping device comprising:
a lower socket, adapted to receive and secure a first portion of a limb in a receptacle of said lower socket;
an upper socket, adapted to receive and secure a second portion of said limb in a receptacle of said upper socket;
an attachment;
a socket connector, positioned at a distal end of said lower socket;
wherein said lower socket and said upper socket are attached to one another and are rotatable with respect to one another;
wherein said attachment is configured to be removably secured to said socket connector at a number of different angles; and
wherein said attachment is configured to receive a tensile force caused by movement of said limb to cause said attachment to apply a gripping force.

10. The device of claim 9, wherein said upper socket is substantially curved in shape, and wherein open space is present opposite of said upper socket.

11. The device of claim 9, further comprising a pair of top edge links, wherein said lower socket includes a top edge having portions proximate to said top edge links curved below a remainder of said top edge.

12. The device of claim 10, further comprising a strap configured to secure said limb to said device.

13. The device of claim 9, further comprising a string configured to apply said tensile force, and a tensioning pin, wherein said tensioning pin is configured to regulate tension in said string.

14. The device of claim 13, further comprising a worm gear, wherein said tensioning pin is configured to regulate tension in said string by regulating motion of said worm gear.

15. The device of claim 9, wherein said attachment comprises a plurality of apertures each configured to be aligned with an aperture of said socket connector to permit a locking object to be positioned through aligned apertures to secure said attachment to said socket connector.

16. A method for assembling an adjustable prosthetic gripping device, the method comprising:
providing a first socket, adapted to receive and secure a portion of a residual arm in a receptacle of said first socket;
providing an attachment;
providing a socket connector, positioned at a distal end of said first socket;
configuring said attachment to be removably secured to said socket connector at a number of different angles; and
configuring said attachment to receive a tensile force caused by movement of said limb to cause said attachment to apply a gripping force.

17. The method of claim 16, further comprising:
providing a second socket, adapted to receive and secure another portion of said limb in a receptacle of said second socket;
attaching said first socket and said second socket to one another;
configuring said first socket and said second socket to be rotatable with respect to one another; and
providing open space opposite of said second socket.

18. The method of claim 17, further comprising providing a curved beam in said first socket.

19. The method of claim 17, further comprising providing a foam interior in each of said first socket and said second socket.

20. The method of claim 16, further comprising providing a string configured to apply said tensile force, wherein said string extends from said attachment through an aperture of said socket connector.
